(19) United States
(12) Patent Application Publication
(10) Pub. No.: US 2023/0201089 A1
(43) Pub. Date: Jun. 29, 2023

(54) AGENTS FOR IMPROVED OXIDATIVE LIGHTENING OF KERATIN FIBRES

(71) Applicant: Henkel AG & Co. KGaA, Duesseldorf (DE)

(72) Inventors: Konstantin GOUTSIS, Juechen (DE); Daniela KESSLER-BECKER, Leverkusen (DE)

(73) Assignee: Henkel AG & Co. KGaA, Duesseldorf (DE)

(21) Appl. No.: 17/916,417

(22) PCT Filed: Jan. 5, 2021
(86) PCT No.: PCT/EP2021/050043

§ 371 Date: Sep. 30, 2022

(30) Foreign Application Priority Data
Mar. 31, 2020 (DE) 10 2020 204 145.8

(51) Int. Cl. A61K 8/22; A61K 8/36; A61K 8/44; A61Q 5/00

(52) U.S. Cl. CPC A61K 8/22; A61K 8/361; A61K 8/44; A61Q 5/00; A61K 2800/4322

(57) ABSTRACT

The present disclosure relates to agents for lightening keratinous fibers, in particular human hair, comprising
(a) at least one oxidizing agent,
(b) at least one complexing agent of the general formula (I),

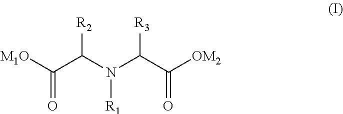

and
(c) at least one radical inhibitor.

### CROSS-REFERENCE TO RELATED APPLICATION

This application is a U.S. National-Stage entry under 35 U.S.C. § 371 based on International Application No. PCT/EP2021/050043, filed Jan. 5, 2021, which was published under PCT Article 21(2) and which claims priority to German Application No. 102020204145.8, filed Mar. 31, 2020, which are all hereby incorporated in their entirety by reference.

### TECHNICAL FIELD

The present disclosure relates to agents for oxidative color change in the field of cosmetics, which are particularly suitable for lightening keratin fibers, especially human hair.

Changing the shape and color of hair is an important area of modern cosmetics. In addition to coloring, lightening one's hair color or bleaching is the specific desire of many consumers, as a blonde hair color is considered attractive and desirable in fashion terms. For this purpose, various blonding agents with different blonding performances are available on the market.

### BACKGROUND

The oxidizing agents included in bleaching agents are able to lighten the hair fiber through the oxidative destruction of the hair's own pigment melanin. For a moderate bleaching effect, the use of hydrogen peroxide—possibly with the addition of ammonia or other alkalizing agents—alone is sufficient as an oxidizing agent; to achieve a stronger bleaching effect, a mixture of hydrogen peroxide and peroxodisulfate salts and/or peroxomonosulfate salts is usually used.

In the case of dark initial hair, longer application times and/or repeated bleaching processes are usually necessary to lighten the hair over several shades. However, this is also accompanied by greater damage to the hair, as not only the hair's colorants, but also the other structural components of the hair are oxidatively damaged. Depending on the degree of damage, this ranges from rough, brittle hair that is more difficult to comb out, to reduced resistance and tensile strength of the hair, to hair breakage.

The use of complexing agents in oxidative color change of keratinic fibers is well known. Among other things, the complexing agents are intended to prevent the decomposition of hydrogen by metal ions accumulated in the hair fibers.

For example, EP 1714634 A1 describes a hair treatment kit for coloring human hair, comprising a first compartment comprising a complexing agent and a second compartment comprising agents for coloring. The use of a complexing agent is intended to prevent undesirable reactions on and with hair that lead to undesirable heating.

Many bleaching products on the market use HEDP (etidronic acid) or its salts or EDTA (ethylenediamine tetraacetate) or its salts as complexing agents. Both HEDP and EDTA effectively stabilize hydrogen peroxide and complex existing metal ions so effectively that an undesired temperature increase occurring during application is almost completely avoided. However, the major disadvantage of HEDP and EDTA is their poor biodegradability. Especially recently, users have been paying increasing attention to the ecological profile of the cosmetic products they use. Thus, the user prefers above all cosmetics that are as sustainable as possible and contain biodegradable ingredients.

It was therefore the task of the present disclosure to find blonding agents with biodegradable complexing agents which, with regard to their blonding performance, are at least comparable to, and preferably even better than, the blonding or brightening agents known from the prior art. Furthermore, the bleaching agents should have a sufficiently high stability and should not heat up too much even when applied to hair with a higher metal or heavy metal content. In addition, the use of complexing agents in lightening or bleaching products should reduce hair damage.

### BRIEF SUMMARY

Agents and processes for lightening keratinous fibers are provided herein. In an embodiment, an agent for lightening keratinous fibers includes:
(a) at least one oxidizing agent,
(b) at least one complexing agent of the general formula (I),

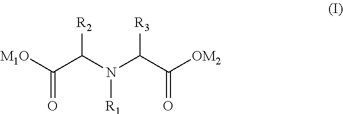

where
R1 represents a hydrogen atom, a C₁-C₆ alkyl group, a hydroxy-C₂-C₆ alkyl group, a carboxy-C₁-C₆alkyl group, or a physiologically acceptable salt thereof,
R2, R3 independently represent a hydrogen atom, a C₁-C₆ alkyl group, a hydroxy-C₂-C₆ alkyl group, a carboxy-C₁-C₆ alkyl group, or a physiologically acceptable salt thereof,
M1, M2 independently of one another represent a hydrogen atom or an equivalent of an alkali metal, alkaline earth metal or metal ion, and
(c) at least one radical inhibitor.

In another embodiment, a process for lightening keratinous fibers includes mixing at least two separately packaged preparations (A) and (B) to form an application mixture, applying the application mixture to the fibers, and rinsing off the application mixture from the fibers after a contact time. The preparation (A) includes hydrogen peroxide (a1). The preparation (B) includes at least one persulfate (a2) selected from the group of ammonium peroxodisulfate, potassium peroxodisulfate or sodium peroxodisulfate. The preparation (B) further includes at least one complexing agent (b) of the general formula (I) as described above and at least one radical inhibitor (c).

In another embodiment, a process for lightening keratinous fibers includes mixing at least three separately packaged preparations (A) and (B) and (C) to form an application mixture, applying the application mixture to the fibers, and rinsing off the application mixture from the fibers after an exposure time. The preparation (A) includes hydrogen peroxide (a1). The preparation (B) includes at least one persulfate (a2) selected from the group of ammonium peroxodisulfate, potassium peroxodisulfate or sodium peroxodisulfate. The preparation (C) includes at least one complexing agent (b) of the general formula (I) as described above, and further includes at least one radical inhibitor (c).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description. It is to be appreciated that all values as provided herein, save for the actual examples, are approximate values with endpoints or particular values intended to be read as “about” or “approximately” the value as recited.

Surprisingly, it has now been found that this task can be fully solved if a lightening or bleaching agent is used which, in addition to at least one oxidizing agent (a), also comprises at least one special complexing agent (b) of a specific formula (I) and a radical scavenger (c).

A first object of the present disclosure is an agent for lightening keratinous fibers, in particular human hair, comprising.
(a) at least one oxidizing agent,
(b) at least one complexing agent of the general formula (I),

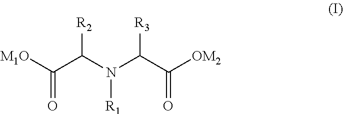

where
R1 represents a hydrogen atom, a C₁-C₆alkyl group, a hydroxy-C₂-C₆ alkyl group, a carboxy-C₁-C₆ alkyl group or a physiologically acceptable salt thereof,
R2, R3 independently represent a hydrogen atom, a C₁-C₆ alkyl group, a hydroxy-C₂-C₆ alkyl group, a carboxy-C₁-C₆ alkyl group or a physiologically acceptable salt thereof,
M1, M2 independently of one another represent a hydrogen atom or an equivalent of an alkali metal, alkaline earth metal or metal ion, preferably sodium, potassium, ½ magnesium, ½ calcium, ½ zinc, or an ammonium ion (NH₄⁺), and
(c) at least one radical inhibitor.

### Keratinous Fibers

Keratinous fibers, or keratin fibers, mean furs, wool, feathers and, in particular, human hair. Although the agents are primarily suitable for lightening keratin fibers, in principle there is nothing to prevent their use in other areas as well.

### Agent for Lightening Keratinous Fibers

The term “lightening of keratin fibers” as used in the present disclosure includes any form of color change of the fibers in which the keratin fibers have a lighter coloration compared to the color present before the application of the agent. This includes in particular the color changes covered under the terms lightening, blonding and bleaching. The lighter coloration of the hair is caused by the oxidizing agent(s) present in the agent. In addition to the oxidizing agent(s), the agents of the present disclosure may also contain colorant components, such as oxidation dye precursors and/or direct dyes, for the purpose of shading. The coloring components can easily modify the color appearance of the resulting coloration. As contemplated herein, however, these colorant components are present in the agent in such small amounts that the color impression of the keratin fibers treated with the agent is nevertheless lighter than their original color. Corresponding coloring techniques can be called coloring bleaching or nuancing bleaching.

The agent comprises the essential ingredients of the present disclosure (a), (b) and (c) preferably in a cosmetic carrier. For example, a suitable aqueous, alcoholic or aqueous-alcoholic carrier can be used as a cosmetic carrier for the agent. For the purpose of hair coloring, such carriers are, for example, creams, emulsions, gels, pastes or also surfactant-comprising foaming solutions, such as shampoos, foam aerosols, foam formulations or other preparations suitable for application to the hair.

Accordingly, the first object of the present disclosure is an agent for lightening keratinous fibers, in particular human hair, comprising in a cosmetic carrier
(a) at least one oxidizing agent,
(b) at least one complexing agent of the general formula (I),

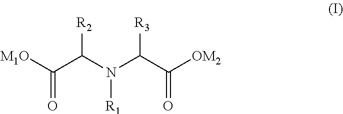

where
R1 represents a hydrogen atom, a C₁-C₆ alkyl group, a hydroxy-C₂-C₆ alkyl group, a carboxy-C₁-C₆ alkyl group or a physiologically acceptable salt thereof,
R2, R3 independently represent a hydrogen atom, a C₁-C₆ alkyl group, a hydroxy-C₂-C₆ alkyl group, a carboxy-C₁-C₆ alkyl group or a physiologically acceptable salt thereof,
M1, M2 independently of one another represent a hydrogen atom or an equivalent of an alkali metal, alkaline earth metal or metal ion, preferably sodium, potassium, ½ magnesium, ½ calcium, ½ zinc, or an ammonium ion (NH₄⁺), and
(c) at least one radical inhibitor.

The agent as contemplated herein is a ready-to-use agent which can be applied in this form to the keratin fibers for the purpose of bleaching or lightening.

### Oxidizing Agent (a)

As a first essential ingredient, the agent as contemplated herein comprises at least one oxidizing agent (a). To achieve moderate whitening effects, hydrogen peroxide is the oxidizing agent of choice. As bleaching and whitening agents, preferred agents include hydrogen peroxide and/or one of its solid addition products to organic or inorganic compounds. However, if a stronger lightening or bleaching effect is desired, hydrogen peroxide is used together with stronger oxidizing agents such as persulfates (sodium persulfate, potassium persulfate or ammonium persulfate).

In the context of a particularly preferred embodiment, an agent as contemplated herein includes

(a) at least one oxidizing agent selected from the group of hydrogen peroxide, ammonium peroxodisulfate, potassium peroxodisulfate or sodium peroxodisulfate.

Ammonium peroxodisulfate, which may alternatively also be referred to as ammonium persulfate, is understood as meaning the persulfate having the empirical formula (NH₄)₂S₂O₈.

Potassium peroxodisulfate, which can alternatively be called potassium persulfate, is the persulfate with the molecular formula K2S2O8.

Sodium peroxodisulfate, which can alternatively be referred to as sodium persulfate, is the persulfate with the molecular formula Na2S2O8.

In the context of a particularly preferred embodiment, an agent as contemplated herein includes
(a1) hydrogen peroxide and
(a2) at least one persulfate selected from the group of ammonium peroxodisulfate, potassium peroxodisulfate or sodium peroxodisulfate.

Accordingly, the first object of the present disclosure is an agent for lightening keratinous fibers, in particular human hair, comprising
(a1) hydrogen peroxide and
(a2) at least one persulfate selected from the group of ammonium peroxodisulfate, potassium peroxodisulfate or sodium peroxodisulfate, and
(b) at least one complexing agent of the general formula (I),

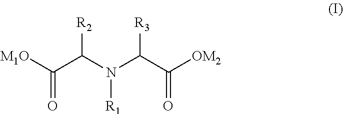

where
R1 represents a hydrogen atom, a C₁-C₆ alkyl group, a hydroxy-C₂-C₆ alkyl group, a carboxy-C₁-C₆ alkyl group or a physiologically acceptable salt thereof,
R2, R3 independently represent a hydrogen atom, a C₁-C₆ alkyl group, a hydroxy-C₂-C₆ alkyl group, a carboxy-C₁-C₆ alkyl group or a physiologically acceptable salt thereof,
M1, M2 independently of one another represent a hydrogen atom or an equivalent of an alkali metal, alkaline earth metal or metal ion, preferably sodium, potassium, ½ magnesium, ½ calcium, ½ zinc, or an ammonium ion (NH₄⁺), and
(c) at least one radical inhibitor.

In a preferred embodiment, hydrogen peroxide itself is used as an aqueous solution. The concentration of a hydrogen peroxide solution in the agent as contemplated herein is determined on the one hand by the legal requirements and on the other hand by the desired effect; preferably 3 to 12 wt. % solutions in water are used. Agents of the first subject matter preferred as contemplated herein are contain, based on the total weight of the agent, (a) from 0.1 to 12.0 wt. % preferably from 0.5 to 10.5 wt. %, further preferably from 1.0 to 8.5 wt. %, still further preferably from 1.5 to 7.0 wt. % and most preferably from 1.5 to 6.0 wt. % hydrogen peroxide.

In the context of a particularly preferred embodiment, an agent as contemplated herein includes
based on the weight of the agent—(a) from 0.1 to 12.0 wt. %, preferably from 0.5 to 10.5 wt. %, more preferably from 1.0 to 8.5 wt. %, still more preferably from 1.5 to 7.0 wt. % and very particularly preferably from 1.5 to 6.0 wt. % of hydrogen peroxide.

The persulfate(s) are also preferably used in certain ranges of amounts in the agent. It has been found to be preferred if the agent comprises, based on the total weight of the agent, (a) one or more persulfates selected from the group of ammonium peroxodisulfate, potassium peroxodisulfate or sodium peroxodisulfate in a total amount of from 2.0 to 40.0% by weight, preferably from 4.0 to 30.0 wt. %, more preferably from 6.0 to 20.0 wt. % and most preferably from 8.0 to 15.0 wt. %.

In the context of a particularly preferred embodiment, an agent as contemplated herein includes
based on the total weight of the agent—(a) one or more persulfates selected from the group of ammonium peroxodisulfate, potassium peroxodisulfate or sodium peroxodisulfate in a total amount of from 2.0 to 40.0 wt. %, preferably from 4.0 to 30.0 wt. %, more preferably from 6.0 to 20.0 wt. % and most preferably from 8.0 to 15.0 wt. %.

Accordingly, in the context of a further embodiment, particularly preferred is an agent for lightening keratinous fibers, in particular human hair, comprising, based on the total weight of the agent
(a1) from 0.1 to 12.0 wt. %, preferably from 0.5 to 10.5 wt. %, more preferably from 1.0 to 8.5 wt. %, still more preferably from 1.5 to 7.0 wt. % and most preferably from 1.5 to 6.0 wt. % of hydrogen peroxide, and
(a2) one or more persulfates selected from the group of ammonium peroxodisulfate, potassium peroxodisulfate or sodium peroxodisulfate in a total amount of from 2.0 to 40 wt. %, preferably from 4.0 to 30.0 wt. %, more preferably from 6.0 to 20.0 wt. % and most preferably from 8.0 to 15.0 wt. %, and
(b) at least one complexing agent of the general formula (I),

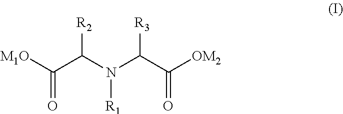

where
R1 represents a hydrogen atom, a C₁-C₆ alkyl group, a hydroxy-C₂-C₆ alkyl group, a carboxy-C₁-C₆ alkyl group or a physiologically acceptable salt thereof,
R2, R3 independently represent a hydrogen atom, a C₁-C₆ alkyl group, a hydroxy-C₂-C₆ alkyl group, a carboxy-C₁-C₆ alkyl group or a physiologically acceptable salt thereof,
M1, M2 independently of one another represent a hydrogen atom or an equivalent of an alkali metal, alkaline earth metal or metal ion, preferably sodium, potassium, ½ magnesium, ½ calcium, ½ zinc, or an ammonium ion (NH₄⁺), and
(c) at least one radical inhibitor.

### Complexing Agent (b)

As a second ingredient essential to the present disclosure, the agent as contemplated herein comprises at least one complexing agent (b) of the general formula (I),

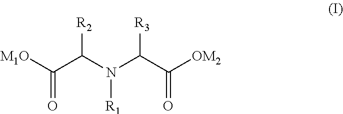

where
R1 represents a hydrogen atom, a C₁-C₆ alkyl group, a hydroxy-C₂-C₆ alkyl group, a carboxy-C₁-C₆ alkyl group or a physiologically acceptable salt thereof,
R2, R3 independently represent a hydrogen atom, a C₁-C₆ alkyl group, a hydroxy-C₂-C₆ alkyl group, a carboxy-C₁-C₆ alkyl group or a physiologically acceptable salt thereof, and
M1, M2 independently of one another represent a hydrogen atom or an equivalent of an alkali metal, alkaline earth metal or metal ion, preferably sodium, potassium, ½ magnesium, ½ calcium, ½ zinc, or an ammonium ion (NH₄⁺).

The complexing agents of formula (I) are described as biodegradable and therefore represent an ecologically advantageous substitute for HEDP and EDTA. Surprisingly, it was found that the use of these special complexing agents (b) in the agent of the present disclosure also massively improved the bleaching performance.

Examples of the substituents R1, R2 and R3 mentioned in formula (I) are given below:

Examples of C₁-C₆-alkyl radicals include —CH₃, —CH₂CH₃, —CH₂CH₂CH₃, —CH(CH₃)₂, —CH₂CH(CH₃)₂, —CH(CH₃)CH₂CH₃, —C(CH₃)₃, —(CH₂)₄CH₃, —(CH₂)₅CH₃. Particularly preferred alkyl radicals are methyl and ethyl. Examples of C₁-C₆-hydroxyalkyl groups include —CH₂OH, CH₂CH₂OH, —CH₂CH₂CH₂OH, —CH₂CH(OH)CH₃, —CH₂CH₂CH₂CH₂OH, with —CH₂CH₂OH being preferred.
Examples of carboxy-C₁-C₆ alkyl groups are HOOC—CH₂—, HOOC—CH₂—CH₂—, HOOC—CH₂—CH₂—CH₂—, HOOC—CH₂—CH₂—CH₂—CH₂—, where the group HOOC—CH₂— is preferred.

In the complexing agents of the general formula (I), the radical R1 is a hydrogen atom, a C₁-C₆-alkyl group, a hydroxy-C₂-C₆-alkyl group, a carboxy-C₁-C₆-alkyl group or a physiologically tolerated salt of a carboxy-C₁-C₆-alkyl group.

Physiologically compatible salts are those salts that can be used in cosmetics under physiological conditions without adverse effects. Examples of a physiologically acceptable salt of a carboxy-C₁-C₆ alkyl group include the sodium salt, the potassium salt and the ammonium salt of the carboxy-C₁-C₆ alkyl group.

A particularly strong improvement in the bleaching performance was observed with the agents comprising at least one complexing agent (b) of the general formula (I), where R1 is a hydrogen atom, a carboxy-C₁-C₆-alkyl group or a physiologically acceptable salt thereof, preferably a hydrogen atom, a carboxymethyl group or a physiologically acceptable salt thereof.

In the context of a particularly preferred embodiment, an agent as contemplated herein includes

at least one complexing agent (b) of the general formula (I), wherein
R1 represents a hydrogen atom, a carboxy-C₁-C₆ alkyl group or a physiologically acceptable salt thereof, preferably a hydrogen atom, a carboxymethyl group or a physiologically acceptable salt thereof.

In the complexing agents of the general formula (I), R2 and R3 independently represent a hydrogen atom, a C₁-C₆ alkyl group, a hydroxy-C₂-C₆ alkyl group, a carboxy-C₁-C₆ alkyl group or a physiologically acceptable salt thereof.

Furthermore, particularly good results were obtained in blonding tests when at least one complexing agent (b) of the formula (I) was used in the agent as contemplated herein, in which the radicals R2 and R3 independently of one another represent a hydrogen atom, a methyl group, a carboxymethyl group or a physiologically tolerated salt thereof.

In the context of a particularly preferred embodiment, an agent as contemplated herein includes

at least one complexing agent (b) of the general formula (I), wherein
R2, R3 independently represent a hydrogen atom, a methyl group, a carboxymethyl group or a physiologically acceptable salt thereof.

The best bleaching results were obtained with complexing agents (b) of formula (I), in which

R1 is a carboxymethyl group or a physiologically acceptable salt thereof,
R2 represents a methyl group and
R3 represents a hydrogen atom.

In the context of a particularly preferred embodiment, an agent as contemplated herein includes

at least one complexing agent (b) of the general formula (I), wherein
R1 is a carboxymethyl group or a physiologically acceptable salt thereof,
R2 represents a methyl group and
R3 represents a hydrogen atom.

An explicit very particularly preferred complexing agent (b) of this embodiment is N,N-bis(carboxymethyl)-L-alanine, which may alternatively be referred to as 2-methyl-2′,2″,2′″-nitrilotriacetic acid and abbreviated as substance MGDA. MGDA has the CAS number 29578-05-0 and can be purchased commercially from various suppliers, for example, from the company ABClabtory Scientific Co. Ltd, from the company Chemieliva Pharmaceutical Co. Ltd, from the company SIA “Chemspace” or from the company Hong Kong Chemhere Co. Ltd, MGDA has the formula (I-a). The physiologically compatible salts of MGDA are also as contemplated herein.

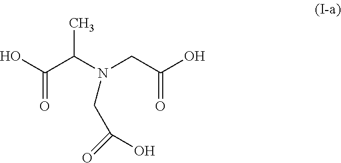

Furthermore, very good bleaching results were obtained with complexing agents (b) of formula (I), where

R1 represents a hydrogen atom,
R2 and R3 are independently a carboxymethyl group or a physiologically acceptable salt thereof.

In the context of a further particularly preferred embodiment, an agent as contemplated herein includes at least one complexing agent (b) of the general formula (I), wherein

R1 represents a hydrogen atom,
R2 and R3 are independently a carboxymethyl group or a physiologically acceptable salt thereof.

An explicitly very particularly preferred complexing agent (b) of this embodiment is aspartic acid-N-(2,3-dicarboxyethyl)tetrannotrium salt, which may alternatively be referred to as tetrasodium iminodissucinate and has CAS number 144538-83-0. This complexing agent is commercially available from Lanxess under the trade name Baypure CX 100. Aspartic acid-N-(2,3-dicarboxyethyl)tetrantrium salt) has the structural formula (I-b)

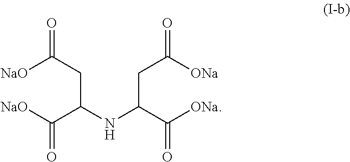

In compounds of formula (I-b), the M1 and M2 radicals both represent a sodium cation. Both the tetrasodium salt and other salts, such as the tetra potassium salt, as well as the tetra carboxylic acid itself, are also as contemplated herein and are very particularly preferred.

Another suitable complexing agent of formula (I) is nitrilotriacetic acid (NTA). In the case of nitrilotriacetic acid, the radical R1 represents a

R1 is a carboxymethyl group or a physiologically acceptable salt thereof, and
R2, R3 both represent a hydrogen atom.

To optimize the bleaching or lightening effect, the complexing agent(s) (b) is/are preferably used in certain ranges of amounts in the agent as contemplated herein.

Tests with the prior art complexing agent HEDP (etidronic acid, CAS No. 2809-21-4) have shown that an increase in the amount of HEDP used does not necessarily lead to an improvement in the blonding performance. With HEDP, therefore, the blonding performance cannot be improved precisely by increasing the feed rate.

Against the background of the known results for HEDP, it was not to be expected that other quantity-activity ratios would exist for the complexing agents (b) as contemplated herein. Now, however, it was surprisingly found that the complexing agents (b) as contemplated herein further improve the blonde effect when higher application amounts are used in the agent.

It is therefore particularly preferred if the agent comprises—based on the total weight of the agent—one or more complexing agents (b) of the formula (I) in a total amount of from 0.05 to 10.0% by weight, preferably from 0.2 to 7.5 wt. %, more preferably from 1.0 to 5.0 wt. % and most preferably from 1.1 to 4.5 wt. %.

In the context of a particularly preferred embodiment, an agent as contemplated herein includes
based on the total weight of the agent—one or more complexing agents (b) corresponding to formula (I) in a total amount of 0.05 to 10.0 wt. %, preferably 0.2 to 7.5 wt. %, more preferably 1.0 to 5.0 wt. % and very particularly preferably 1.1 to 4.5 wt. %.

### Radical Inhibitor (c)

By using a complexing agent (b) as contemplated herein, it was possible to replace the complexing agents HEDP or EDTA, which are commonly used in many market products, without any loss of blonding performance. However, the experiments leading to the present disclosure have shown that the use of the complexing agents (b) alone can lead to higher hair damage and, in addition, also has a negative effect on the temperature profile of the bleaching agents. Thus, in tests on strands, it was shown that bleaching agents comprising the complexing agent (b) alone heated up much more when applied to hair contaminated with metals than corresponding bleaching agents comprising HEDP or EDTA. When applied to the head, a bleaching agent comprising the complexing agent (b) alone may therefore overheat considerably.

In further experiments, it has now been found that the addition of at least one radical inhibitor (c) both minimizes hair damage and greatly reduces the temperatures that develop when the agent comes into contact with metal-comprising hair. For this reason, the agent as contemplated herein comprises at least one radical inhibitor (c) as a third ingredient essential to the present disclosure.

The lightening or bleaching process that takes place on the keratin fiber occurs mainly via radical reactions, with the hydroxyl radical formed from hydrogen peroxide and/or persulfates playing a central role. Radicals are atoms or molecules with at least one unpaired valence electron. As a rule, radicals have a very high reactivity.

For the purposes of the present disclosure, a radical inhibitor refers to substances that react with reactive radicals, such as hydroxyl radicals, and convert them to less reactive species through a series of rapid reactions, thereby interrupting the radical chain reaction.

The radical inhibitors suitable for use in cosmetics can be assigned to specific groups. The use of one or more radical inhibitors from the group of antioxidants, thiols, quinones and unsaturated organic compounds has proved particularly suitable for use in the lightening or bleaching compositions of the present disclosure.

In another particularly preferred embodiment, an agent as contemplated herein includes at least one radical inhibitor (c) selected from the group of unsaturated organic compounds, antioxidants, quinones or thiols.

It has proved to be particularly preferred to use at least one radical inhibitor (c) selected from the group of unsaturated organic compounds in the agent of the present disclosure to solve the problem of the present disclosure. An unsaturated organic compound is understood to be an organic compound with at least one carbon-carbon double bond.

The use of at least one mono- or polyunsaturated C₈-C₃₀ fatty acid and/or the derivative of a mono- or polyunsaturated C₈-C₃₀ fatty acid has proven to be particularly advantageous.

As contemplated herein, unsaturated fatty acids are mono- or polyunsaturated, unbranched or branched, unsubstituted or substituted C₈-C₃₀ carboxylic acids. Unsaturated fatty acids can be mono- or polyunsaturated. For an unsaturated fatty acid, its C—C double bond(s) may have the Cis or Trans configuration.

In another particularly preferred embodiment, an agent as contemplated herein includes at least one radical inhibitor (c) selected from the group of unsaturated C₈-C₃₀ fatty acids or derivatives thereof.

Examples of a C₈-C₃₀ unsaturated fatty acid include petroselinic acid [(Z) octadecenoic acid], palmitoleic acid [(9Z)-hexadec-9-enoic acid], oleic acid [(9Z)-octadec enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid] and/or nervonic acid [(15Z)-tetracos-15-enoic acid]. Very particularly preferred unsaturated C₈-C₃₀ fatty acids are linoleic acid [(9Z,12Z)-octadeca-9,12-dienoic acid, linolenic acid [(9Z,12Z,15Z)-octadeca-9,12,15-trienoic acid.

In another particularly preferred embodiment, an agent as contemplated herein includes at least one radical inhibitor (c) selected from the group of petroselinic acid [(Z)-6-octadecenoic acid], palmitoleic acid [(9Z)-hexadec-9-enoic acid], oleic acid [(9Z)-octadec-9-enoic acid], Elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid] and/or nervonic acid [(15Z)-tetracos-15-enoic acid]. Very particularly preferred unsaturated C₈-C₃₀ fatty acids are linoleic acid [(9Z,12Z)-octadeca-9,12-dienoic acid, linolenic acid [(9Z,12Z,15Z)-octadeca-9,12,15-trienoic acid.

Examples of particularly suitable unsaturated C₈-C₃₀ fatty acids are linoleic acid and linolenic acid. These are essential fatty acids, also known as vitamin F in the literature.

In another very particularly preferred embodiment, an agent as contemplated herein includes at least one radical inhibitor (c) selected from the group of linoleic acid or linolenic acid.

Particularly well-suited derivatives of C₈-C₃₀ fatty acids are their esters.

Characteristic of an ester of a C₈-C₃₀ unsaturated fatty acid is the presence of an ester functional group obtained by esterification of the acid function of the fatty acid with an alcohol. This alcohol may be a monohydric alcohol or a polyhydric alcohol, which may optionally also carry further functional groups or substituents.

Examples of monohydric alcohols include methanol, ethanol, n-propanol, iso-propanol, n-butanol, n-pentanol, n-hexanol, n-heptanol, n-octanol, n-nonanol, n-decanol, n-undecanol, n-dodecanol, n-tetradecanol, n-hexadecanol, and n-octadecanol.

Examples of polyhydric alcohols are 1,2-propanediol, 1,3-propanediol and glycerol. Glycerol is particularly preferred.

Other examples of the esters of C₈-C₃₀ unsaturated fatty acids include corresponding fatty acid monoglycerides, fatty acid diglycerides, and fatty acid triglycerides.

A C₈-C₃₀ fatty acid monoglyceride is the monoester of the trihydric alcohol glycerol with one equivalent of unsaturated C₈-C₃₀ fatty acid. Either the middle hydroxy group of glycerol or the terminal hydroxy group of glycerol may be esterified with the fatty acid.

The C₁₂-C₃₀ fatty acid monoglycerides in which a hydroxy group of glycerol is esterified with a fatty acid, the fatty acids being selected from petroselinic acid [(Z)-6-octadecenoic acid], palmitoleic acid [(9Z)-hexadec-9-enoic acid], oleic acid [(9Z)-octadec-9-enoic acid], Elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], or nervonic acid [(15Z)-tetracos-15-enoic acid].

A C₁₂-C₃₀ fatty acid diglyceride is understood to be the diester of the trivalent alcohol glycerol with two equivalents of C₈-C₃₀ fatty acid, where at least one fatty acid must be an unsaturated C₈-C₃₀ fatty acid. Either the middle and one terminal hydroxy group of glycerol may be esterified with two equivalents of fatty acid, or both terminal hydroxy groups of glycerol are esterified with one fatty acid each. The glycerol can be esterified with two fatty acids of the same structure or with two different fatty acids, with the proviso that at least one fatty acid is an unsaturated C₈-C₃₀ fatty acid.

Fatty acid diglycerides are particularly suitable in which at least one of the ester groups is formed from glycerol with a fatty acid selected from petroselinic acid [(Z) octadecenoic acid], palmitoleic acid [(9Z)-hexadec-9-enoic acid], oleic acid [(9Z)-octadec enoic acid], Elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], or nervonic acid [(15Z)-tetracos-15-enoic acid].

A C₁₂-C₃₀ fatty acid triglyceride is understood to be the triester of the trivalent alcohol glycerol with three equivalents of C₈-C₃₀ fatty acid, where at least one fatty acid must be an unsaturated C₈-C₃₀ fatty acid. The glycerol can be esterified with three fatty acids of the same structure or with different fatty acids, with the proviso that at least one fatty acid is an unsaturated C₈-C₃₀ fatty acid.

Fatty acid triglycerides are particularly suitable in which at least one of the ester groups is formed from glycerol with a fatty acid selected from petroselinic acid [(Z)-6-octadecenoic acid], palmitoleic acid [(9Z)-hexadec-9-enoic acid], oleic acid [(9Z)-octadec-9-enoic acid], Elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], or nervonic acid [(15Z)-tetracos-15-enoic acid].

Explicitly, radical inhibitors (c) from the group of phosphoglycerides have proved to be particularly suitable for solving the problem of the present disclosure. Phosphoglycerides fall under the group of esters of C₈-C₃₀ fatty acids.

In this context, phosphoglycerides as contemplated herein are understood to be substances that can alternatively also be referred to as glycerophospholipids/phosphoglycerolipids.

Phosphoglycerides in the sense of the present disclosure are built up from glycerol esterified with two C₈-C₃₀ fatty acids at two of the hydroxy groups (OH groups) of the glycerol, with the proviso that at least one fatty acid is an unsaturated C₈-C₃₀ fatty acid. A phosphate group is attached to one of the third, terminal OH groups. This phosphate group is in turn esterified with different alcohols. The phosphate group forms a phosphoric acid diester.

Explicitly, radical inhibitors (c) from the lecithin cucumber are particularly preferred.

In the context of a further explicitly quite particularly preferred embodiment, an agent as contemplated herein includes at least one radical inhibitor (c) of the general formula (II)

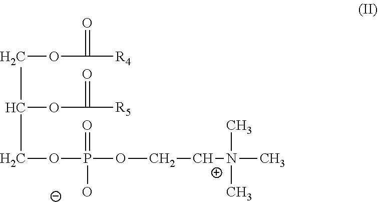

where
R4, R5 independently represent a saturated or unsaturated C₁₁-C₂₉ alkyl group, with the proviso that at least one of R4 and R5 represents an unsaturated C₁₁-C₂₉ alkyl group.

Preferably, the R4 and/or R5 radicals may represent the following radicals. Together with the carbonyl group adjacent to the R4 or R5 radical, this radical forms the esterified form of the fatty acid given in the table:

| Fatty acid | R4 and/or R5 |
|---|---|
| Petroselinic acid [(Z)-6-octadecenoic acid]. | 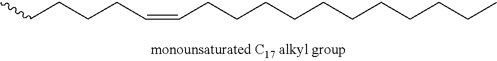 |
| Palmitoleic acid [(9Z)-Hexadec-9- acid] | 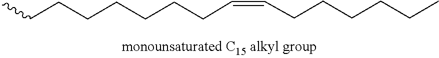 |
| Oleic acid [(9Z)-Octadec-9- acid] | 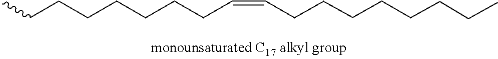 |
| Elaidinous acid [(9E)-Octadec-9- acid] | 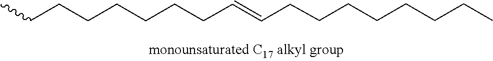 |
| Erucic acid [(13Z)-Docos-13- acid] | 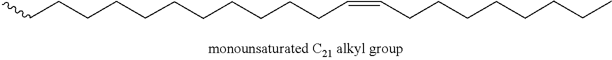 |
| Linoleic acid [(9Z,12Z)-Octadeca- 9,12-di acid | 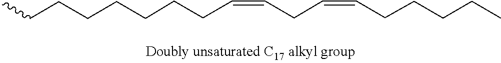 |
| Linolenic acid [(9Z,12Z,15Z)- Octadeca-9,12,15- tri acid | 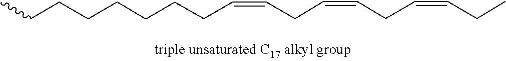 |
| Elaeostearic acid [(9Z,11E,13E)- Octadeca-9,11,3- tri acid] | 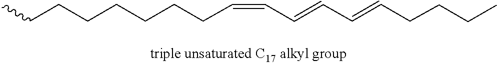 |
| Arachidonic acid [(5Z,8Z,11Z,14Z)- Icosa-5,8,11,14- tetra acid] | 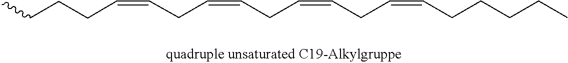 |
| Nervonic acid [(15Z)-Tetracos- 15-acid] | 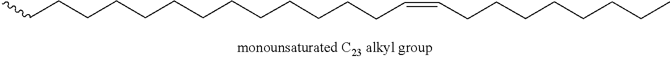 |

Preferably, at least one of the radicals R4 and/or R5 stands for an at least monounsaturated C₁₆-C₂₀-alkyl group, particularly preferably for an at least diunsaturated C₁₆-C₂₀-alkyl group and explicitly most preferably for an at least triunsaturated C₁₆-C₂₀-alkyl group.

In another explicitly very particularly preferred embodiment, an agent as contemplated herein includes at least one radical inhibitor (c) of the general formula (II), where at least one of the radicals R4 and/or R5 is an at least monounsaturated C₁₅-C₂₁-alkyl group, particularly preferably an at least diunsaturated C₁₅-C₂₁-alkyl group and explicitly very particularly preferably an at least triunsaturated C₁₅-C₂₁-alkyl group.

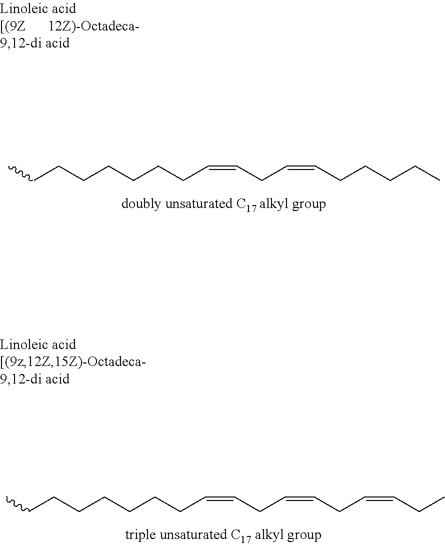

In the context of a further explicitly quite particularly preferred embodiment, an agent as contemplated herein includes at least one radical inhibitor (c) of the general formula (II), where at least one of the radicals R4 and/or R5 represents a is a doubly unsaturated C₁₇ alkyl group or a triply unsaturated C₁₇ alkyl group.

If one of the remaining R4 or R5 is a saturated C₁₁-C₂₉ alkyl group, this group may preferably be a saturated C₁₁ alkyl group, a saturated C₁₃ alkyl group, a saturated C₁₅ alkyl group, a saturated C₁₇ alkyl group, a saturated C₁₉ alkyl group or a saturated C₂₁ alkyl group.

A particularly suitable lecithin of formula (II) is soybean lecithin, which is commercially available under the trade name Lipoid P20 from the company Lipoid GmbH. This raw material has a phosphatidylcholine (=lecithin) content of at least 20% by weight, at least 61% by weight of the fatty acids—based on the total content of fatty acids obtained after hydrolysis of the lecithin—being linoleic acid or linolenic acid.

As radical inhibitors (c) from the group of antioxidants, vitamin F, vitamin E, vitamin C, vitamin A, vitamin B and vitamin H have proven to be particularly suitable.

Vitamin F: The term vitamin F, as described above, usually refers to essential fatty acids, especially linoleic acid, linolenic acid.

Vitamin E: Vitamin E is a collective term for fat-soluble substances with mostly antioxidant effects. The most abundant forms of vitamin E are called tocopherols and tocotrienols. As a particularly well-suited vitamin E, α-tocopherol can be used. α-Tocopherol is alternatively known as (2R)-2,5,7,8-tetramethyl-2-[(4R,8R)-4,8,12-trimethyltridecyl]-3,4-dihydro-2H-chromen-6-ol or as E 307 and carries the CAS number 10191-41-0.

The group of substances known as vitamin A includes retinol (vitamin A1) and 3,4-didehydroretinol (vitamin A2). The β-carotene is the provitamin of retinol. As contemplated herein, vitamin A components that can be considered are, for example, vitamin A acid and its esters, vitamin A aldehyde and vitamin A alcohol as well as its esters such as palmitate and acetate.

Also suitable as contemplated herein are vitamin C (ascorbic acid) and its esters, in particular ascorbyl palmitate.

The vitamin B group or vitamin B complex includes, among others.
Vitamin B₁ (Thiamine)
Vitamin B₂ (Riboflavin)
Vitamin B₃. The compounds nicotinic acid and nicotinamide (niacinamide) are frequently listed under this designation, among which nicotinamide in particular is preferred as contemplated herein.
Vitamin B5 (pantothenic acid and panthenol). Within this group, panthenol is preferred. Derivatives of panthenol that can be used as contemplated herein are, in particular, the esters and ethers of panthenol, cationically derivatized panthenols and pantolactone.
Vitamin B₆ (pyridoxine as well as pyridoxamine and pyridoxal).

Vitamin H. The compound (3aS,4S,6aR)-2-oxohexahydrothienol[3,4-d]-imidazole-4-valeric acid is known as vitamin H, but the trivial name biotin has since become established.

In the context of a further preferred embodiment, an agent as contemplated herein includes at least one radical inhibitor (c) selected from the group of vitamin F, vitamin E, vitamin C, vitamin A, vitamin B, or vitamin H.

In the context of a further particularly preferred embodiment, an agent as contemplated herein includes at least one radical inhibitor (c) selected from the group of vitamin F, vitamin E, vitamin C and vitamin A.

Compounds from the quinone group are suitable as radical inhibitors (c). Quinones are organic compounds that have a quinoid system. Suitable representatives from the group of quinones are 1,4-benzoquinone, 1,4-naphthoquinone, 2-hydroxy-1,4-naphthoquinone. Also suitable are 1,4-benzoquinones and 1,4-napthoquinones bearing at least one substituent such as a hydroxyl group, an amino group, a nitro group, a carboxylic acid group, a C₁-C₆ alkyl group, a C₁-C₆ alkoxy group.

Examples of suitable radical inhibitors (c) from the group of thiols are thiolactic acid, ammonium thioglycolate and ammonium thiolactate.

Thiolactic acid is understood to mean D-thio-lactic acid, L-thio-lactic acid and/or a mixture thereof. Cysteine is understood to mean D-cysteine, L-cysteine and/or their mixture.

Ammonium thioglycolate is the ammonium salt of thiglycolic acid (i.e. the ammonium salt of sulfanyl acetic acid) (formula Thiol-I)

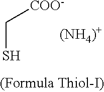

Ammonium thiolactate is the ammonium salt of thiolactic acid (i.e. the ammonium salt of 2-sulfanylpropionic acid) (formula thiol-II).

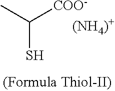

The definition of ammonium thiolactate includes both the ammonium salts of D-thiolactic acid and the salts of L-thiolactic acid and mixtures thereof.

To optimize the desired effect, the radical inhibitors (c) are also preferably used in the agent of the present disclosure in certain ranges of amounts. It has been found to be particularly advantageous if the agent comprises—based on the total weight of the agent—one or more radical inhibitors (c) in a total amount of from 0.01 to 10.0 wt. %, preferably from 0.25 to 7.0 wt. %, more preferably from 0.25 to 6.0 wt. % and most preferably from 0.25 to 2.5 wt. %.

In a further preferred embodiment, an agent as contemplated herein includes—based on the total weight of the agent—one or more radical inhibitors (c) in a total amount of from 0.01 to 10.0 wt. %, preferably from 0.25 to 7.0 wt. %, more preferably from 0.25 to 6.0 wt. % and very particularly preferably from 0.25 to 2.5 wt. %.

### Alkalizing Agent

As previously described, the agent of the first present disclosure as contemplated herein is an application-ready agent for lightening or bleaching keratin fibers. In order to achieve a sufficient bleaching effect, such agents are usually strongly alkaline, the pH value preferably being between 9 and 10.5. Such high pH values are necessary to ensure opening of the outer cuticle layer and thus allow penetration of the active species (hydrogen peroxide and persulfates) into the hair.

For this reason, it has been found to be particularly preferred if the agent as contemplated herein additionally comprises at least one alkalizing agent.

In the context of a further preferred embodiment, an agent as contemplated herein includes at least one alkalizing agent.

Preferred alkalizing agents are, for example, ammonia, alkanolamines, basic amino acids, and inorganic alkalizing agents such as (earth) alkali metal hydroxides, (earth) alkali metal metasilicates, (earth) alkali metal silicates, (earth) alkali metal phosphates and (earth) alkali metal hydrogen phosphates. Preferred metal ions are lithium, sodium and/or potassium. Preferred alkalizing agents are (earth) alkali metal metasilicates and (earth) alkali metal silicates.

Suitable inorganic alkalizing agents are preferably selected from sodium hydroxide, potassium hydroxide, calcium hydroxide, barium hydroxide, sodium phosphate, potassium phosphate, sodium silicate, potassium silicate, magnesium silicate, sodium carbonate and potassium carbonate. Sodium hydroxide and/or potassium hydroxide are particularly preferred.

Alkanolamines as alkalizing agents are preferably selected from primary, secondary or tertiary amines having a C₂-C₆ alkyl parent bearing at least one hydroxyl group. Particularly preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methyl-propanol, 2-amino-2-methylbutanol, 3-amino-2-methyl propan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine methylglucamine, triethanolamine, diethanolamine and triisopropanolamine. Particularly preferred alkanolamines are monoethanolamine, 2-amino-2-methyl-propanol and triethanolamine.

Basic amino acids as alkalizing agents are preferably selected from the group formed by L-arginine, D-arginine, D/L-arginine, L-lysine, D-lysine, D/L-lysine, L-ornithine, D-ornithine, D/L-ornithine, L-histidine, D-histidine and/or D/L-histidine. L-arginine, D-arginine and/or D/L-arginine are particularly preferred as an alkalizing agent.

### Waiver of HEDP or EDTA

The aim of the present application is in particular to dispense with the non-biodegradable complexing agents HEDP and EDTA. For this reason, the agents as contemplated herein preferably contain these two complexing agents in particularly small amounts. Very preferably, the agents are free of these two substances. Most preferably, the agent is also substantially free of the salts of HEDP and EDTA.

In a further preferred embodiment, an agent as contemplated herein includes—based on the total weight of the agent—the total content of etidronic acid and of the salts of etidronic acid included in the agent is below 0.2 wt. %, preferably below 0.1 wt. %, more preferably below 0.05 wt. % and very particularly preferably below 0.001 wt. %.

In a further preferred embodiment, an agent as contemplated herein includes—based on the total weight of the agent—the total content of EDTA and the salts of EDTA included in the agent is below 0.2% by weight, preferably below 0.1 wt. %, further preferably below 0.05 wt. % and very particularly preferably below 0.001 wt. %.

Etidronic acid is alternatively known as hydroxyethane-1,1-diphosphonic acid and has the CAS number 2809-21-4.

EDTA is also known as ethylenediamine tetraacetate and has the CAS numbers 6381-92-6 and 139-33-3 (anhydrous).

### Other Ingredients

In addition to the ingredients (a), (b) and (c) essential to the present disclosure—and optionally the alkalizing agents—the agent as contemplated herein may also contain other active ingredients and auxiliaries as non-mandatory components. These are described below.

The agents may also contain other active ingredients, auxiliaries and additives, such as solvents, fatty components such as C₈-C₃₀ fatty alcohols, C₈-C₃₀ fatty acid triglycerides, C₈-C₃₀ fatty acid monoglycerides, C₈-C₃₀ fatty acid diglycerides and/or hydrocarbons; nonionic surfactants, anionic surfactants, cationic surfactants, amphoteric and/or zwitterionic surfactants, polymers; structurants such as glucose, maleic acid and lactic acid, hair conditioning compounds such as phospholipids, for example lecitin and cephalism; perfume oils, dimethyl isosorbide and cyclodextrins; fiber structure-improving agents, in particular mono-, di- and oligosaccharides such as glucose, galactose, fructose, fructose and lactose; dyes for coloring the agent; antidandruff agents such as piroctone olamine, zinc omadine and climbazole; amino acids and oligopeptides; Protein hydrolysates based on animals and/or plants, as well as in the form of their fatty acid condensation products or, if appropriate, anionically or cationically modified derivatives; vegetable oils; light stabilizers and UV blockers; Active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, plant extracts; Fats and waxes such as fatty alcohols, beeswax, montan wax and kerosenes; swelling and penetrating agents such as glycerol, propylene glycol monoethyl ether, carbonates, hydrogen carbonates, guanidines, ureas as well as primary, secondary and tertiary phosphates; pacifiers such as latex, styrene/PVP and styrene/acrylamide copolymers; pearlescent agents such as ethylene glycol mono- and distearate and PEG-3 distearate; and blowing agents such as propane-butane mixtures, N₂O, dimethyl ether, CO₂ and air.

The selection of these other substances will be made by the specialist according to the desired properties of the agents. With regard to other optional components and the quantities of these components used, explicit reference is made to the relevant manuals known to the specialist. The additional active ingredients and auxiliary substances are preferably used in the preparations as contemplated herein in quantities of 0.0001 to 25 wt. % each, in particular 0.0005 to 15 wt. %, based on the total weight of the respective agent.

### Keratin Fiber Lightening Process

As previously described, the agent of the first subject matter of the present disclosure represents the ready-to-use lightening or bleaching agent which, in addition to the oxidizing agent(s) (a), also comprises complexing agent(s) (b), radical inhibitor(s) (c) and, optionally, alkalizing agent(s).

Oxidizing agents such as hydrogen oxide and persulfates are highly reactive compounds that have limited stability, especially in alkaline environments. For this reason, the ready-to-use blonding agent is usually prepared just before use by mixing two or more separately packaged preparations.

Usually, the oxidizing agents (a) and the alkalizing agents are prepared separately. For the complexing agent (b) and the radical inhibitor (c), different types of packaging are now conceivable.

For example, complexing agents (b) and the radical inhibitors (c) can be made up together with one or more persulfates and separately from hydrogen peroxide. This embodiment is particularly preferred when only two different preparations are to be mixed together to prepare the agent ready for use.

A further object of the present disclosure is a process for lightening keratinous fibers, wherein at least two separately packaged preparations (A) and (B) are mixed to form an application mixture, which is applied to the fibers and rinsed off again after an exposure time, wherein
the preparation (A) comprises hydrogen peroxide (a1), and
the preparation (B) comprises
at least one persulfate (a2) selected from the group of ammonium peroxodisulfate, potassium peroxodisulfate or sodium peroxodisulfate, and
comprises at least one complexing agent (b) of the general formula (I) and
comprises at least one radical inhibitor (c),
   wherein the complexing agent (b) and the radical inhibitor (c) have already been disclosed in detail in the description of the first subject matter of the present disclosure.

Preparations (A) and (B) may be mixed either with each other only or with other separately packaged preparations immediately prior to use to form an application mixture.

If three different preparations are to be mixed together to produce the ready-to-use agent, it may be preferable to provide the first oxidizing agent hydrogen peroxide separately in a first preparation (A), to provide the persulfates as the second oxidizing agent separately in a second preparation (B), and furthermore to provide a third preparation (C) comprising complexing agent (b) and the radical inhibitor (c).

A further object of the present disclosure is a process for lightening keratinous fibers, wherein at least three separately packaged preparations (A) and (B) and (C) are mixed to form an application mixture, which is applied to the fibers and rinsed off again after an exposure time, wherein
the preparation (A) comprises hydrogen peroxide (a1), and
the preparation (B) comprises at least one persulfate (a2) selected from the group of ammonium peroxodisulfate, potassium peroxodisulfate or sodium peroxodisulfate, and
the preparation (C) comprises at least one complexing agent (b) of the general formula (I) and comprises at least one radical inhibitor (c),
   wherein the complexing agent (b) and the radical inhibitor (c) have already been disclosed in detail in the description of the first subject matter of the present disclosure.

The preparations (B) comprising the persulfates are preferably in powder form. Powders of solid components with different particle sizes can be used. Typically, however, it may be preferred if the powders have a particle size that is as homogeneous as possible, in particular to facilitate uniform dispersion or dissolution of the powders in the preparations (B).

Further in, the preparations (B) may also contain the persulfates in a solid cosmetic carrier. A solid cosmetic carrier may contain salts of silicic acid, in particular salts of silicates and metasilicates with ammonium, alkali metals as well as alkaline earth metals. In particular, metasilicates have a ratio between n and m of 1 according to the formula (SiO₂)ₙ(M₂O)ₘ, where M stands for an ammonium ion, an alkali metal or a half stoichiometry equivalent of an alkaline earth metal, and can be understood as chain-like polymeric structures of the anion [SiO₃]²—, can be preferably used. sodium metasilicate of the formula [Na₂SiO₃]_{∞}, is particularly preferred. Equally preferred are those silicates formed from a silicate of the formula (SiO₂)ₙ(Na₂O)ₘ(K₂O)ₚ, where n is a positive rational number and m and p are independently a positive rational number or 0, with the provisos that at least one of the parameters m or p is other than 0 and the ratio between n and the sum of m and p is between 2:1 and 4:1.

Furthermore, the solid preparations (B) may contain so-called anti-caking agents, which are intended to prevent the powder constituents from clumping or caking. Preferably, water-insoluble, hydrophobic or moisture-adsorbing powders of diatomaceous earth, pyrogenic silica, calcium phosphate, calcium silicates, aluminum oxide, magnesium oxide, magnesium carbonate, zinc oxide, stearates, fatty amines and the like are suitable as such anti-caking agents.

Finally, preparations (B) may additionally contain a dedusting agent that prevents dust formation of the powdered components. Inert oils in particular can be used for this purpose. Preferably, the solid cosmetic carriers contain ester oils or mineral oils, preferably hydrocarbon oils, such as liquid kerosene oil, as dusting agents.

The ready-to-use agents are prepared immediately before application to the hair by mixing the two preparations (A) and (B) or by mixing the three preparations (A) and (B) and (C). For ready-to-use agents that are mixed from more than two preparations to form a finished application mixture, it may be irrelevant whether two preparations are first mixed together and then the third preparation is added and mixed in, or whether all preparations are combined together and then mixed. Mixing can be done by stirring in a bowl or cup, or by shaking in a sealable container.

The term “immediate” is to be understood as a period of time from a few seconds to one hour, preferably up to 30 min, in particular up to 15 min.

Preparations (A), (B) and optionally (C) are used in a process for lightening keratinous fibers, in particular human hair, in which the agent is applied to the keratinous fibers, left on the fibers for a period of 10 to 60 minutes and then rinsed out again with water or washed out with a shampoo.

Preferably, the exposure time of the ready-to-use brightening agents is 10 to 60 min, in particular 15 to 50 min, especially preferably 20 to 45 min. During the exposure time of the agent on the fiber, it may be advantageous to support the brightening process by applying a small amount of heat. The heat can be supplied by an external heat source, such as with the aid of a hot air blower, or, especially in the case of hair lightening on living subjects, by the body temperature of the subject. With the latter option, the area to be lightened is usually covered with a cap. An exposure phase at room temperature is preferred.

After the end of the exposure time, the remaining lightening preparation is rinsed out of the hair with water or a cleansing agent. In particular, commercially available shampoo can be used as a cleaning agent, although the cleaning agent can be dispensed with and the rinsing process can be carried out with tap water if the whitening agent has a carrier with a high surfactant content.

The preferred embodiments of the agent described above apply mutatis mutandis to the method.

### EXAMPLES

### 1. Preparation of the Ready-to-Use Whitening Agent by Mixing Three Preparations (A), (B) and (C)

The following preparations were prepared (all data in wt. % unless otherwise stated).

| Composition (A) | Wt. % |
|---|---|
| Disodium pyrophosphate | 0.1 |
| Dipicolinic acid | 0.1 |
| Potassium hydroxide (50% aqueous solution) | 0.3 |
| Cetearyl alcohol | 3.6 |
| Ceteareth-20 | 0.5 |
| Sodium lauryl sulfate | 0.3 |
| PEG-40 Castor Oil | 0.6 |
| Isopropyl myristate | 10.0 |
| Hydrogen peroxide, 50% aqueous solution | 23.2 |
| Water | ad 100 |

| Preparation (B) | Wt. % |
|---|---|
| Potassium persulphate | 98.4 |
| Silica (fumed) | 1.4 |

| | C1 | C2 | C3 | C4 | C5 |
|---|---|---|---|---|---|
| Preparation (C) | Comparison | Comparison | Comparison | Comparison | Comparison |
| Cetearyl alcohol | 5.75 | 5.75 | 5.75 | 5.75 | 5.75 |
| Lorol techn. (C12-C18 | 2.7 | 2.7 | 2.7 | 2.7 | 2.7 |
| fatty alcohols) | | | | | |
| Ceteareth-20 | 0.25 | 0.25 | 0.25 | 0.25 | 0.25 |
| Sodium laureth sulfate | 6 | 6 | 6 | 6 | 6 |
| (C12-14, 2EO 27% | | | | | |
| aqueous solution) | | | | | |
| Ammonium sulfate | 0.5 | 0.5 | 0.5 | 0.5 | 0.5 |
| Ammonia 25% | 7.6 | 7.6 | 7.6 | 7.6 | 7.6 |
| Sodium silicate 40/42 | 0.5 | 0.5 | 0.5 | 0.5 | 0.5 |
| Potassium hydroxide 50% | 1 | 1 | 1 | 1 | 1 |
| HEDP 60% | 0.2 | 3.0 | — | — | 3.0 |
| MGDA Powder | — | — | 0.2 | 3.0 | — |
| Vitamin F | — | — | — | — | 3.0 |
| Lipoid P 20 | — | — | — | — | — |
| Water | ad 100 | ad 100 | ad 100 | ad 100 | ad 100 |

MGDA: N,N-Bis(carboxymethyl)-L-alanine, CAS No. 29578-05-0
Vitamin F: LINOLEIC ACID, LINOLENIC ACID (Polichimica), CAS No. 1106-87-4
Lipoid P20: Soybean Lecithin, CAS No. 8030-76-0, Lipoid GmbH Ludwigshafen

| | C6 | C7 | C8 | C9 | C10 |
|---|---|---|---|---|---|
| Preparation (C) | Comparison | Invention | Invention | Invention | Invention |
| Cetearl alcohol | 5.75 | 5.75 | 5.75 | 5.75 | 5.75 |
| Lorol techn. 12-C18 | 2.7 | 2.7 | 2.7 | 2.7 | 2.7 |
| fatty alcohols) | | | | | |
| Ceteareth-20 | 0.25 | 0.25 | 0.25 | 0.25 | 0.25 |
| Sodium laureth sulfate | 6 | 6 | 6 | 6 | 6 |
| (C12-14, 2EO 27% | | | | | |
| aqueous solution) | | | | | |
| Ammonium sulfate | 0.5 | 0.5 | 0.5 | 0.5 | 0.5 |
| Ammonia 25% | 7.6 | 7.6 | 7.6 | 7.6 | 7.6 |
| Sodium silicate 40/42 | 0.5 | 0.5 | 0.5 | 0.5 | 0.5 |
| Potassium hydroxide 50% | 1 | 1 | 1 | 1 | 1 |
| HEDP 60% | 3.0 | — | — | — | — |
| MGDA Powder | — | 0.2 | 0.2 | 3.0 | 3.0 |
| Vitamin F | — | 0.2 | — | 3.0 | — |
| Lipoid P 20 | 3.0 | — | 0.2 | — | 3.0 |
| Water | ad 100 | ad 100 | ad 100 | ad 100 | ad 100 |

### 2. Dyeing

Hair strands (Kerling, Euronaturhaar 4-0) were measured colorimetrically (Datacolor Spectraflash SF 450), pre-shampooed and dried.

To prepare the ready-to-use brightening or bleaching agent, in each case 60 g of the preparation (A) were mixed with 20 g of the preparation (B) and 60 g of the preparation (C). The resulting application mixture was applied to the hair strands, left there for 45 minutes and then rinsed again with water. Afterwards, the hair strands were measured colorimetrically again.

The higher the ΔL value, the greater the lightening of the strands compared to untreated hair.

The higher the ΔE value, the greater the color shift of the strands compared to the untreated hair.

| Application mixture | L | a | b | C | h | ΔL | ΔE | ΔC |
|---|---|---|---|---|---|---|---|---|
| (A) + (B) + (C1) | 37.71 | 10.75 | 22.51 | 24.94 | 64.48 | 17.83 | 26.52 | 19.63 |
| 0.2% HEDP | | | | | | | | |
| (A) + (B) + (C1) | 38.88 | 10.54 | 22.69 | 25.02 | 65.09 | 19.00 | 27.38 | 19.72 |
| 0.2% HEDP | | | | | | | | |
| (A) + (B) + (C2) | 36.69 | 10.93 | 21.51 | 24.13 | 63.07 | 16.81 | 25.19 | 18.77 |
| 3% HEDP | | | | | | | | |
| (A) + (B) + (C2) | 38.13 | 11.16 | 23.40 | 25.93 | 64.50 | 18.25 | 27.52 | 20.61 |
| 3% HEDP | | | | | | | | |
| (A) + (B) + (C3) | 39.86 | 11.47 | 24.48 | 27.03 | 64.89 | 19.98 | 29.51 | 21.73 |
| 0.2% MGDA | | | | | | | | |
| (A) + (B) + (C3) | 39.70 | 11.14 | 24.40 | 26.83 | 65.46 | 19.82 | 29.27 | 21.53 |
| 0.2% MGDA | | | | | | | | |
| (A) + (B) + (C4) | 44.57 | 11.29 | 27.59 | 29.82 | 67.75 | 24.69 | 34.85 | 24.60 |
| 3% MGDA | | | | | | | | |
| (A) + (B) + (C4) | 44.53 | 10.92 | 27.01 | 29.13 | 67.99 | 24.65 | 34.36 | 23.93 |
| 3% MGDA | | | | | | | | |
| (A) + (B) + (C5) | 38.62 | 11.73 | 24.47 | 27.14 | 64.40 | 18.74 | 28.75 | 21.81 |
| 3% HEDP + 3% Vitamin F | | | | | | | | |
| (A) + (B) + (C5) | 38.18 | 11.15 | 22.91 | 25.48 | 64.06 | 18.30 | 27.22 | 20.15 |
| 3% HEDP + 3% Vitamin F | | | | | | | | |
| (A) + (B) + (C6) | 37.28 | 11.04 | 21.60 | 24.26 | 62.94 | 17.40 | 25.68 | 18.89 |
| 3% HEDP + 3% Lipoid P 20 | | | | | | | | |
| (A) + (B) + (C6) | 36.40 | 10.81 | 21.24 | 23.83 | 63.01 | 16.52 | 24.78 | 18.47 |
| 3% HEDP + 3% Lipoid P 20 | | | | | | | | |
| (A) + (B) + (C8) | 42.57 | 11.12 | 25.49 | 27.81 | 66.43 | 33.74 | 31.99 | 22.55 |
| 0.2% MGDA + 0.2% LipoidP20 | | | | | | | | |
| (A) + (B) + (C8) | 41.34 | 11.18 | 24.6 | 27.02 | 65.55 | 33.85 | 30.54 | 21.74 |
| 0.2% MGDA + 0.2% LipoidP20 | | | | | | | | |
| (A) + (B) + (C9) | 44.31 | 11.68 | 28.57 | 30.86 | 67.76 | 24.43 | 35.42 | 25.65 |
| 3% MGDA + 3% Vitamin F | | | | | | | | |
| (A) + (B) + (C9) | 43.56 | 11.37 | 27.07 | 29.36 | 67.21 | 23.68 | 33.81 | 24.13 |
| 3% MGDA + 3% Vitamin F | | | | | | | | |
| (A) + (B) + (C10) | 40.85 | 11.39 | 25.74 | 28.14 | 66.14 | 20.97 | 31.03 | 22.88 |
| 3% MGDA + 3% Lipoid P 20 | | | | | | | | |
| (A) + (B) + (C10) | 42.66 | 11.60 | 26.88 | 29.27 | 66.66 | 22.78 | 33.11 | 24.02 |
| 3% MGDA + 3% Lipoid P 20 | | | | | | | | |

### 3. Measurement of the Temperature Curve During Brightening

Strands with high metal content were used to measure the temperatures occurring during bleaching. For the production of this hair strands were treated as follows:

Hair strands (Kerling, Euronaturhaar white, 6-0) were pre-shampooed and then pre-blonded once with a commercial bleaching agent. Pre-blonding was done to increase copper absorption in the hair. The strand thus pre-blonded was doped with copper:

For this purpose, 1 g strand at a time was immersed in 40 g of a copper solution (50 ppm copper in water, 16.8° dH) and left there for 1 minute. Then the strand was removed from the solution, lightly squeezed and stocked at room temperature. For each strand, this dipping process was repeated three times.

The copper content of each hair strand was then determined analytically. The determination was made after mineralization of the sample (one strand at a time) in the microwave digestion system with HNO3/HCL using atomic emission spectrometry (ICP-OES). The mean value was calculated from 12 measurements. The mean copper content of each hair strand was 3900 mg/kg.

The application mixtures described in point 2 were applied to the hair treated in this way. The strands were fitted with a Pt100 probe and wrapped in aluminum foil. The reaction temperature was measured and documented every 5 minutes.

Temperature (° C.)

| Comparison | Comparison | Minutes |
|---|---|---|
| (A) + (B) + (C2) | (A) + (B) + (C4) | Reaction |
| 3% HEDP | 3% MGDA | time |
| 37.1 | 40.2 | 5 |
| 33.6 | 37.9 | 10 |
| 31.8 | 34.7 | 15 |
| 30.5 | 32.5 | 20 |

| Temperature (° C.) | | | | |
|---|---|---|---|---|
| Comparison | | Invention | | |
| (A) + (B) + (C6) | (A) + (B) + (C5) | (A) + (B) + (C10) | (A) + (B) + (C9) | Minutes |
| 3% HEDP | 3% HEDP | 3% MGDA | 3% MGDA | Reaction |
| 3% Lipoid P20 | 3% Vitamin F | 3% Lipoid P20 | 3% Vitamin F | time |
| 36.0 | 36.5 | 35.3 | 35.1 | 5 |
| 35.6 | 35.0 | 33.9 | 32.2 | 10 |
| 33.2 | 33.2 | 31.9 | 31.3 | 15 |
| 30.5 | 30.4 | 29.1 | 29.8 | 20 |

The application mixtures as contemplated herein became less warm during application and therefore showed a more favorable temperature profile.

After 45 minutes, the application mixture was rinsed from the respective strand of hair with water. Then the strands of hair were dried.

### 4. Measurement of the Cysteic Acid Content

The hair strands lightened in point 3 were used to measure the hair damage. Hair damage of hair lightened as previously described was determined by quantitative NIR spectroscopy.

The spectra were recorded with an MPA™ FT-NIR Spetkrometer from Bruker Optik GmbH. The infrared region covers the wavenumber range from 12500 cm⁻¹ to 4000 cm⁻¹ and is characteristic of overtone and combination vibrations of e.g. CH, OH and NH groups.

The measurement of the samples was carried out with the intercalation sphere module at six different sample positions in diffuse reflection. For the analysis of the measured NIR spectra, the wavenumber ranges from 7300 cm⁻¹ to 4020 cm⁻¹ was chosen.

The NIR spectra of cysteine show characteristic absorption bands in the wavenumber range from 6200 cm⁻¹ to 5500 cm⁻¹. If the hair changes due to more severe damage (i.e., the cysteic acid content in the hair increases), this affects the bands characteristic of cysteic acid at 5020 cm⁻¹ to 4020 cm⁻¹ in the NIR spectrum. The quantitative evaluation of the NIR spectra was computer-aided.

NIR analysis value [mol cysteic acid/100 mol amino acid].

| | mol cysteic acid/ | |
|---|---|---|
| Application mixture | 100 mol amino acid | |
| (A) + (B) + (C2) | 5.4 | Comparison |
| 3% HEDP | | |
| (A) + (B) + (C4) | 5.6 | |
| 3% MGDA | | |
| (A) + (B) + (C5) | 5.9 | |
| 3% HEDP + 3% Vitamin F | | |
| (A) + (B) + (C6) | 5.6 | |
| 3% HEDP + 3% Lipoid P 20 | | |
| (A) + (B) + (C9) | 5.3 | Invention |
| 3% MGDA + 3% Vitamin F | | |
| (A) + (B) + (C10) | 5.2 | |
| 3% MGDA + 3% Lipoid P 20 | | |

Reduced hair damage was measured with the application mixtures as contemplated herein.

### 5. Further Formulation Examples

Preparation of the ready-to-use whitening agent by mixing two preparations (A) and (B).

| Composition (A) | Wt. % |
|---|---|
| Disodium pyrophosphate | 0.1 |
| Dipicolinic acid | 0.1 |
| Potassium hydroxide (50% aqueous solution) | 0.3 |
| Cetearyl alcohol | 3.6 |
| Ceteareth-20 | 0.5 |
| Sodium lauryl sulfate | 0.3 |
| PEG-40 Castor Oil | 0.6 |
| Isopropyl myristate | 10.0 |
| Hydrogen peroxide, 50% aqueous solution | 23.2 |
| Water | ad 100 |

| Preparation (B) (wt. %) | B1 | B2 | B3 | B4 |
|---|---|---|---|---|
| Sodium silicate | 33.0 | 33.0 | 33.0 | 33.0 |
| Magnesium carbonate | 12.8 | 12.8 | 12.8 | 12.8 |
| Sodium hexametaphosphate | 0.2 | 0.2 | 0.2 | 0.2 |
| Degalan RG S mv (Methyl | 1.0 | 1.0 | 1.0 | 1.0 |
| methacrylate, methacrylic | | | | |
| acid copolymer, Evonik) | | | | |
| Polyquaternium-4 | 0.3 | 0.3 | 0.3 | 0.3 |
| Silica (fumed) | 0.4 | 0.4 | 0.4 | 0.4 |
| Potassium persulphate | 32.0 | 32.0 | 32.0 | 32.0 |
| Ammonium persulphate | 10.0 | 10.0 | 10.0 | 10.0 |
| Ariabel Blue 300302 | 0.2 | 0.2 | 0.2 | 0.2 |
| Dimethicone, Dimethiconol | 1.5 | 1.5 | 1.5 | 1.5 |
| MGDA | 0.5 | 1.0 | 2.0 | 3.0 |
| Vitamin F | 0.5 | 1.0 | 2.0 | 3.0 |
| Lipoid P 20 | — | — | — | — |
| Paraffinum Liquidum | ad 100 | ad 100 | ad 100 | ad 100 |

| Preparation (B) (wt. %) | B5 | B6 | B7 | B8 |
|---|---|---|---|---|
| Sodium silicate | 33.0 | 33.0 | 33.0 | 33.0 |
| Magnesium carbonate | 12.8 | 12.8 | 12.8 | 12.8 |
| Sodium hexametaphosphate | 0.2 | 0.2 | 0.2 | 0.2 |
| Degalan RG S mv (Methyl | 1.0 | 1.0 | 1.0 | 1.0 |
| methacrylate, methacrylic | | | | |
| acid copolymer, Evonik) | | | | |
| Polyquaternium-4 | 0.3 | 0.3 | 0.3 | 0.3 |
| Silica (fumed) | 0.4 | 0.4 | 0.4 | 0.4 |
| Potassium persulphate | 32.0 | 32.0 | 32.0 | 32.0 |
| Ammonium persulphate | 10.0 | 10.0 | 10.0 | 10.0 |
| Ariabel Blue 300302 | 0.2 | 0.2 | 0.2 | 0.2 |
| Dimethicone, Dimethiconol | 1.5 | 1.5 | 1.5 | 1.5 |
| MGDA | 0.5 | 1.0 | 2.0 | 3.0 |
| Vitamin F | — | — | — | — |
| Lipoid P 20 | 0.5 | 1.0 | 2.0 | 3.0 |
| Paraffinum Liquidum | ad 100 | ad 100 | ad 100 | ad 100 |

By mixing preparation (A) with one of the preparations (B1) to (B8) each, a ready-to-use bleaching agent was prepared.

| Preparation (B) (% by weight) | B9 | B10 | B11 | B12 |
|---|---|---|---|---|
| Sodium silicate | 36.0 | 36.0 | 36.0 | 36.0 |
| Magnesium carbonate | 10.6 | 3.8 | 7.2 | 7.2 |
| Sodium hexametaphosphate | 0.2 | 0.2 | 0.2 | 0.2 |
| Hydroxyethyl cellulose | 0.4 | 0.4 | 0.4 | 0.4 |
| Cellulose gum | 2.0 | 2.0 | 2.0 | 2.0 |
| MGDA | 1.6 | 5.0 | 1.6 | 5.0 |
| Lipoid P 20 | 1.6 | 5 | 5 | 1.6 |
| Silica (fumed) | 0.4 | 0.4 | 0.4 | 0.4 |
| Ariabel Blue 300302 | 0.2 | 0.2 | 0.2 | 0.2 |
| Potassium persulfate | 32.0 | 32.0 | 32.0 | 32.0 |
| Ammonium persulphate | 10.0 | 10.0 | 10.0 | 10.0 |
| Perfume | 0.6 | 0.6 | 0.6 | 0.6 |
| Paraffinum Liquidum | ad 100 | ad 100 | ad 100 | ad 100 |

By mixing preparation (A) with one of the preparations (B9) to (B12) each, a ready-to-use bleaching agent was prepared.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. An agent for lightening keratinous fibers comprising
(a) at least one oxidizing agent,
(b) at least one complexing agent of the general formula (I), 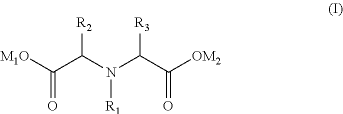
where
R1 represents a hydrogen atom, a C₁-C₆ alkyl group, a hydroxy-C₂-C₆ alkyl group, a carboxy-C₁-C₆alkyl group, or a physiologically acceptable salt thereof,
R2, R3 independently represent a hydrogen atom, a C₁-C₆ alkyl group, a hydroxy-C₂-C₆ alkyl group, a carboxy-C₁-C₆ alkyl group, or a physiologically acceptable salt thereof,
M1, M2 independently of one another represent a hydrogen atom or an equivalent of an alkali metal, alkaline earth metal or metal ion, and
(c) at least one radical inhibitor.

2. The agent according to claim 1, wherein the at least one oxidizing agent (a) is selected from the group of hydrogen peroxide, ammonium peroxodisulfate, potassium peroxodisulfate or sodium peroxodisulfate.

3. Agent according to claim 1, wherein the at least one oxidizing agent (a) comprises:
(a1) hydrogen peroxide and
(a2) at least one persulfate selected from the group of ammonium peroxodisulfate, potassium peroxodisulfate or sodium peroxodisulfate.

4. An agent according to claim 1, comprising—based on the weight of the agent—from 0.1 to 12.0 wt. % of hydrogen peroxide as the at least one oxidizing agent (a).

5. An agent according to claim 1, comprising—based on the total weight of the agent—one or more persulfates selected from the group of ammonium peroxodisulfate, potassium peroxodisulfate or sodium peroxodisulfate in a total amount of from 2.0 to 40.0 wt. % as the at least one oxidizing agent (a).

6. An agent according to claim 1, wherein in the at least one complexing agent (b) of the general formula (I),
R1 represents a hydrogen atom, a carboxy-C₁-C₆ alkyl group or a physiologically acceptable salt thereof.

7. An agent according to claim 1, wherein in the at least one complexing agent (b) of the general formula (I),
R2, R3 independently represent a hydrogen atom, a methyl group, a carboxymethyl group or a physiologically acceptable salt thereof.

8. An agent according to claim 1, wherein in the at least one complexing agent (b) of the general formula (I),
R1 is a carboxymethyl group or a physiologically acceptable salt thereof,
R2 represents a methyl group and
R3 represents a hydrogen atom.

9. An agent according to claim 1, wherein in the at least one complexing agent (b) of the general formula (I),
R1 represents a hydrogen atom,
R2 and R3 are independently a carboxymethyl group or a physiologically acceptable salt thereof.

10. An agent according to claim 1, comprising—based on the total weight of the agent—one or more complexing agents (b) of the formula (I) in a total amount of from 0.05 to 10.0 wt. %.

11. An agent according to claim 1, wherein the at least one radical inhibitor (c) is selected from the group of unsaturated organic compounds, antioxidants, quinones or thiols.

12. An agent according to claim 1, wherein the at least one radical inhibitor (c) is selected from the group of unsaturated C₈-C₃₀ fatty acids or derivatives thereof.

13. An agent according to claim 1, wherein the at least one radical inhibitor (c) is selected from the group of petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid] or nervonic acid [(15Z)-tetracos-15-enoic acid].

14. An agent according to claim 1, wherein the at least one radical inhibitor (c) is of the general formula (II) 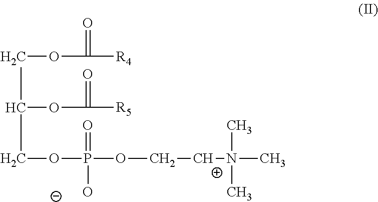
where
R4, R5 independently represent a saturated or unsaturated C₁₁-C₂₉ alkyl group, with the proviso that at least one of R4 and R5 represents an unsaturated C₁₁-C₂₉ alkyl group.

15. The agent according to claim 14, wherein in the at least one radical inhibitor (c) of the general formula (II), at least one of the radicals R4 and/or R5 is an at least monounsaturated C₁₅-C₂₁-alkyl group.

16. An agent according to claim 1, wherein the at least one radical inhibitor (c) is selected from the group of vitamin F, vitamin E, vitamin C, vitamin A, vitamin B or vitamin H.

17. An agent according to claim 1, comprising—based on the total weight of the agent—one or more radical inhibitors (c) in a total amount of from 0.01 to 10.0 wt. %.

18. An agent according to claim 1, comprising at least one alkalizing agent.

19. A process for lightening keratinous fibers, comprising mixing at least two separately packaged preparations (A) and (B) to form an application mixture, applying the application mixture to the fibers, and rinsing off the application mixture from the fibers after a contact time, wherein
the preparation (A) comprises hydrogen peroxide (a1), and
the preparation (B) comprises
at least one persulfate (a2) selected from the group of ammonium peroxodisulfate, potassium peroxodisulfate or sodium peroxodisulfate, and
at least one complexing agent (b) of the general formula (I) as described in claim 1, and
at least one radical inhibitor (c) as described in claim 1.

20. A process for lightening keratinous fibers, comprising mixing at least three separately packaged preparations (A) and (B) and (C) to form an application mixture, applying the application mixture to the fibers, and rinsing off the application mixture front the fibers after an exposure time, wherein
the preparation (A) comprises hydrogen peroxide (a1),
the preparation (B) comprises at least one persulfate (a2) selected from the group of ammonium peroxodisulfate, potassium peroxodisulfate or sodium peroxodisulfate, and
the preparation (C) comprises at least one complexing agent (b) of the general formula (I) as described in claim 1, and further comprises at least one radical inhibitor (c) as described in claim 1.
